(19) United States
(12) Patent Application Publication
(10) Pub. No.: US 2019/0281402 A1
(43) Pub. Date: Sep. 12, 2019

(54) TESTING OF DIRECTIONAL HEARING AS USED IN ACTIVE DRIVER ASSISTANCE SYSTEMS

(71) Applicant: FORD GLOBAL TECHNOLOGIES, LLC, Dearborn, MI (US)

(72) Inventors: Eike Schmidt, Koeln (DE); Abhinav Dhake, Aachen (DE); Stefan Wolter, Würselen (DE)

(21) Appl. No.: 16/278,939

(22) Filed: Feb. 19, 2019

(30) Foreign Application Priority Data
Mar. 12, 2018 (DE) 102018203661.6

(51) Int. Cl. H04S 7/00; H04R 5/04; A61B 5/12; A61B 5/00; B60R 99/00; B60Q 5/00; B60Q 3/70; B60Q 3/80

(52) U.S. Cl. CPC H04S 7/30; H04R 5/04; A61B 5/123; A61B 5/6893; B60R 99/00; G06K 9/00355; B60Q 3/70; B60Q 3/80; H04R 2430/20; H04R 2499/13; B60Q 5/005

(57) ABSTRACT

Apparatus for testing the directional hearing of a test person uses an audio system with a plurality of audio transducers to produce audible audio signals to a position of the test person and a light output system with a plurality of light output devices to produce visible light signals to the position of the test person. A control device is configured to activate the audio system to output a plurality of audible audio signals one after another arriving at the position of the test person in such a way that they arrive or appear to arrive from a selected one of a plurality of different directions, and to activate the light output devices to indicate the selected direction at a particular time in relation to the respective audio signals.

### CROSS REFERENCE TO RELATED APPLICATIONS

This application claims priority to application DE102018203661.6, filed in the German Patent and Trademark Office on Mar. 12, 2018, which is incorporated herein by reference in its entirety.

### STATEMENT REGARDING FEDERALLY SPONSORED RESEARCH

Not Applicable.

### BACKGROUND OF THE INVENTION

The present invention relates to an apparatus for testing and training of the directional hearing of a person involved in operating a vehicle, a corresponding vehicle apparatus, and a method for testing the directional hearing of a person making use of the apparatus.

Modern vehicles (e.g., motor vehicles) are known to have a large number of advanced control, monitoring, and warning systems. In particular, driver assistance systems are known that monitor the surroundings of the vehicle, e.g. the distance from other objects. Amongst other things, such systems may output audible audio signals, such as warning tones, speech information, or other noises or sound, in order to inform the driver about a situation. If a direction is associated with the information (e.g., a dangerously low clearance is detected on a particular side of the vehicle) it can be provided that the audio signal is emitted toward the driver from a direction coincident with the location of the detection.

A collision detection and warning system is disclosed in U.S. Pat. No. 7,046,128B2, wherein obstacles in the blind spot of a vehicle, for example in a neighboring lane, are recognized, and then an alarm tone and/or visual alarm signal is output through the loudspeaker on the side of the vehicle nearest the obstacle. Flashing lights may also be activated on that side.

An automobile alarm system is described in German patent DE10300275A1, wherein a warning location is first detected. Then an associated virtual sound source is determined, so that a warning tone can be generated that is perceived by the person in the vehicle interior as if it came from there.

U.S. Pat. No. 8,686,843B2 discloses a vehicle with a warning system which detects the position of a hazardous location and first generates a directed warning tone that comes from the direction of the hazardous location. The warning system then generates a further warning tone that draws the attention of the driver in another direction, for example, the direction toward which the steering wheel should be turned in order to avoid the hazardous location.

A driver assistance system is presented in US patent application publication 2015/0258930A1, wherein flasher noises or acoustic warning announcements are output together with visual indications to the driver in such a way that they can be assigned either to the left hand or to the right hand side for the driver of the vehicle.

A signaling system for a vehicle is shown in US patent application publication 2007/0210906A1 which recognizes positions of hazardous locations and superimposes a visual warning symbol in the field of view of the driver in such a way that the driver recognizes the direction in which the hazardous location lies from the displayed position of the warning symbol. It can additionally be provided that an acoustic warning signal is output from the loudspeaker of the audio system of the vehicle that lies closest to the direction of the hazardous location.

A warning apparatus for a motor vehicle that detects obstacles in the blind spot of the vehicle and, according to their position, outputs a visual warning in a display field assigned to the direction, is presented in U.S. Pat. No. 8,022,818B2. An audible warning signal can additionally be output.

The driver must, however, in the case of direction-dependent acoustic information, also be functionally able to distinguish the direction from which the relevant audio signal comes from other possible directions from which audio signals, for example from the same or a different driver assistance system, could also come, in order to be able to perceive this information. For some people this capability can be less well developed, and it can drop with increasing age. In order to foster the fastest possible reaction time, the driver is preferably not reliant on additional visual information that shows him the direction.

### SUMMARY OF THE INVENTION

It is an object of the present invention to make available a facility that is suitable for determining or improving the capability of a test person for directional hearing for use in a vehicle.

According to a first aspect of the invention, an apparatus for testing the directional hearing of a test person comprises an audio system, a light output system, and a control device that is connected or can be connected to the audio system and the light output system. The audio system comprises a plurality of audio output devices for the output of audible audio signals to be heard at a position of a test person. The light output system comprises a plurality of light output devices for the output of light, to be seen at least at the position of the test person and indicative of a plurality of different directions. During a testing/training session (e.g., when a vehicle is not being actively driven), the control device is designed on the one hand to arrange for the audio system to sequentially output a plurality of audible audio signals one after another to the position of the test person in such a way that, for the test person, an apparent or actual audio signal source location of the respective audio signal can be varied among a plurality of different directions. On the other hand, the control device is designed to arrange for the light output system to assign at least one of the light output devices to each of the plurality of different directions in which the audio signal source can be located for the test person, and to arrange for these, depending on the audio signal being output at the particular time, to output light at least also at the position of the test person.

In this way an apparatus is made available with which audio signals can be played to a test person from different directions, wherein for each audio signal a light signal, perceptible by the test person, is output or emitted, the signal also identifying the direction from which the audio signal came. If the sequence and direction of the source of the audio signal is not known in advance to the test person, they can thus test or train their directional hearing to recognize the direction from which the audio signal came and can check their conjecture on the basis of the associated light signal.

The selection of the audio signals used for a test can, for example, be made to depend on the field of application of the apparatus for which the directional hearing is to be trained. If an apparatus for use in the interior of a vehicle is involved, the warning tones, speech information, or other noises or sounds whose capture is relevant to the driver of the vehicle can in particular be used as audio signals.

When used in a vehicle interior, the test person is in particular the driver of the vehicle whose position is in a driver seat. The apparatus can, however, also be used for training or testing the directional hearing of a person in the passenger seat or in one of the rear seats in the interior of the vehicle. The “position of the test person” includes not only the location coordinates, but also the orientation.

The audio signal source as used herein is the place from which the sound waves of the audio signal appear to originate according to the perception of a test person, regardless of whether they are truly generated there (i.e., whether it is a primary, secondary, or virtual source). The different directions in which an audio signal source can be located refer herein to their position with respect to the position of the test person with an unchanged direction of view, for example, a driver of a vehicle with a steady view to the front.

An audio system preferably comprises two or more audio output devices arranged with a spatial separation, which can in particular be comprised of loudspeakers or other electro-acoustic transducers. An audio system can, in addition, comprise further devices in order to control the audio output devices. In a vehicle, the audio system can, for example, be the vehicle's own sound installation for an audio entertainment system or mobile communication system, with loudspeakers distributed over various positions in the vehicle interior or its walls.

A light output system preferably comprises two or more light output devices (e.g., lights, lamps, light emitting diodes (LEDs) or other lighting means) that output or emit/radiate visible light when in an active state. In addition, a light output system comprises, amongst other things, further devices to control the light output devices.

To output light depending on the respective audio signal being output at the particular time refers in particular to a time-dependency, and therefore implies that the light is output after the end of a corresponding audio signal, in particular after a significant delay. The light output can also involve outputting the light simultaneously with the audio signal, for example in an initial phase, to clarify the relationship between audio and light signals for the test person.

The functionality of the control device can, in one embodiment, be realized as a separate unit, for example in a logic circuit intended for this purpose or a programmable device, with the aid of program code that is present in the memory for this purpose. In another embodiment, the functionality can also, for example, be realized through programmable devices, e.g. of the audio system, or of the light output system, or, in the case of a vehicle, in another control device of the vehicle.

In a preferred embodiment, the audio system is a directional audio system that comprises means for generating the audio signal sources as virtual audio signal sources created by driving a plurality of audio output devices simultaneously in various ways to simulate the plurality of different directions. This can, for example, involve an already existing “directional audio system” (i.e., an audio system already designed for the determination and output of directed sound or directional audio, e.g., on board a vehicle), wherein the control device of the apparatus for testing directional hearing must above all communicate the desired position of the audio signal source to the audio system. In a further embodiment, a part or the whole of the computing functionality for realizing a “directional audio system” with the loudspeakers of an audio system can be contained in the control device of the apparatus for testing directional hearing. The means for generating the virtual audio signal source therefore comprise, depending on the embodiment, in addition to the audio output devices, at least for example one programmable device in order to be able to determine the virtual audio signal source itself, or, for example, circuits with which received parameter values describing the audio signal source can be used for driving the audio output devices.

The determination of the virtual audio signal source herein includes the case in which the position of the generated virtual audio signal source accords with one of the real audio output devices, and therefore in fact only the real audio output device, i.e. one of the loudspeakers, is directly driven.

The use of a directional audio system offers the advantage that a large number of different directions can be included in this way in the training, even when a real loudspeaker is not arranged precisely in each of the directions. Since highly accurate positions of obstacles or hazardous locations are also in some circumstances determined in driver assistance systems in the vehicle, a capability for more accurately distinguishing the audio signal directions by the driver is also desirable. The use of virtual audio signal sources also offers the advantage that tests or training sequences can be optimized in directional regions in which it is most likely that a plurality of audio warning signals are to be expected, but that differing audio signal sources can also be computed without having to make a change to the existing loudspeakers of the audio system.

In a further exemplary embodiment, the control device is designed to generate the virtual audio signal source as a moving virtual audio signal source. In this way, the directional hearing can also be tested under realistic conditions in situations in which the test apparatus and/or object emitting noises in the surroundings of the apparatus locally move. This embodiment is thus in particular suitable for training directional hearing in driving situations, e.g. in a motor vehicle.

In another embodiment, the audio system comprises means for selecting one of the plurality of audio output devices at a time as the audio signal source. Instead of determining a virtual audio signal source, the selection of possible directions from which the audio signal can come is reduced to the number of audio output devices or loudspeakers arranged spatially at different locations. It is an advantage here that the drive of the audio output devices may be reduced to activating the selected output device and arranging for the audio signal to be output through it.

It is again true for this embodiment, that either the control device only controls the test operation, that is, for example, indicates the position from which the sound of the audio signal should come, while the audio system itself implements the realization, or that the control logic can be realized in the control device and the audio system comprises primarily the loudspeakers themselves.

In a preferred embodiment of the device for testing the directional hearing of a test person, the plurality of audio output devices are loudspeakers of an audio system for the interior of a vehicle.

In an exemplary embodiment, the plurality of light output devices then comprises a row of lights in the interior of the vehicle, for example in the form of a (for example flexible) light strip or of a (possibly entirely or only partially closed) ring of lights, attached or attachable to the sides, or to some sides, and/or to the roof region of the interior of the vehicle. The lighting means used for this, i.e., the light output devices, can, for example, be LEDs, but can also be other lamps or lights. A plurality of lighting devices can thus be attached in a simple manner, each of which has a different angle with respect to the position and alignment of the test person, and also, when virtual audio signal sources that are generated with a directional audio system are used, a light output device in the same or at least a similar direction as each calculated audio signal source can thus be assigned thereto with good accuracy.

In a preferred embodiment the control device is designed to arrange for the light output system to output the light with a delay with respect to the audio signal being output at the particular time. It is preferably provided here that the delay is large enough, for example 2 seconds or longer, for the test person to make their estimate of the direction from which they have heard the audio signal truly only on the basis of what has been heard, and that the light, output after a delay, serves only for monitoring or confirmation. In a further embodiment it can be provided that the light output is delayed for monitoring until it is enabled by a trigger signal, for example with the aid of a speech recognition system and an audio command from the test person.

In one embodiment, the control device is designed to specify the different directions for the audio signals to be output by the audio system one after another at least approximately randomly. A random generator is, for example, used for this purpose. In this way it is ensured that the test person does not, for example when the apparatus is used frequently, adapt to the sequence of audio signal directions and thus reduce the training effect. In another embodiment, however, specific test sequences may also be provided which, for example, ensure that all the directions are called up within a specific period of time, or that specific directions from which, for example, particularly urgent warning signals are to be expected. In a vehicle, for example, the warning of an obstacle in the blind spot of the driver would be preferentially called up.

In yet another embodiment, the apparatus further comprises an evaluation device with a camera sensor and a gesture recognition module for recognizing a direction in which the test person is pointing. In this embodiment, the test person does not themselves decide whether they have correctly recognized the direction of the audio signal, but indicates through a gesture, for example with a finger, a hand, an arm or a rotation of the head, where they suppose the direction of the audio source to be. The gesture detection module comprises an image analysis software and a programmable device in order to recognize and evaluate the gestures recorded with the camera sensor. When used in a vehicle, the camera sensor can, for example, be an already existing camera with which the state of the driver is observed, in order to recognize tiredness.

According to a second aspect of the invention, a vehicle comprises an apparatus for testing the directional hearing of a test person according to the first aspect of the invention. In this way, the advantages and special features of the apparatus according to the invention for testing the directional hearing of a test person are also implemented in the context of a vehicle.

In a preferred embodiment, the apparatus is designed to capture a driving state of the vehicle and only to permit an output of the audio signals and light signals when stationary or when the vehicle is in a self-controlling mode (i.e., self-driving mode of an autonomous vehicle). In this way it is ensured that the apparatus is only used for testing or training when the driver does not need all his attention for the road traffic.

According to a third aspect of the invention, a method for testing the directional hearing of a test person with an apparatus according to the first aspect of the invention comprises, in a first step, a selection of a direction unknown to a test person relative to their position. In a second step, the method comprises an output, with an audio system, of an audible audio signal that comes from a direction unknown to the test person. In a third step, an output of light then takes place at least also at the position of the test person with a light output device of a light output system, wherein the light output device is assigned to indicate the direction of the audio signal. The apparatus is operated in this way, through the output of an audio signal from a direction unexpected by the test person and a subsequent light signal, to make it possible for the test person to estimate the direction of what has been heard and then to check this estimation on the basis of the light signal.

In a preferred embodiment, the selection of the direction unknown to the test person relative to their position, the output of the audio signal, and the output of the light are repeated at least once. The training effect is thus increased through the training with at least two audio signals (and more preferably longer sequences of audio signals) from directions that in each case are unexpected by the test person. It is to be noted that, when repeating, the previously unknown direction is now known, and for repeating the first step a different unknown direction is thus selected.

In one embodiment, it is in particular provided that the output of the light is delayed with respect to the output of the audio signal. In this way it is ensured that the estimation of the direction from which the audio signal comes is based only on what has been heard by the test person, and the output light signal serves only for checking, whereby better training results can be achieved.

In one embodiment, the method furthermore comprises a renewed output of the audio signal and the simultaneous renewed output of the light. Each audio signal is repeated simultaneously with the light signal, preferably with a time delay, to check and improve the learning effect.

In one preferred embodiment, the selection of the direction unknown to the test person relative to their position takes place with the use of a random generator. In this way it is ensured in a simple manner that the selected direction is, every time, a direction that is unknown, i.e., unexpected, for the test person.

### BRIEF DESCRIPTION OF THE DRAWINGS

FIG. 1 is a schematic illustration of an example of an apparatus for testing the directional hearing of a test person according to a first embodiment of the invention.

FIG. 2 is a schematic illustration of an example of a vehicle with an apparatus for testing the directional hearing of a test person according to a second embodiment of the invention.

FIG. 3 is a flowchart of a method for testing the directional hearing of a test person according to a third embodiment of the invention.

### DETAILED DESCRIPTION OF PREFERRED EMBODIMENTS

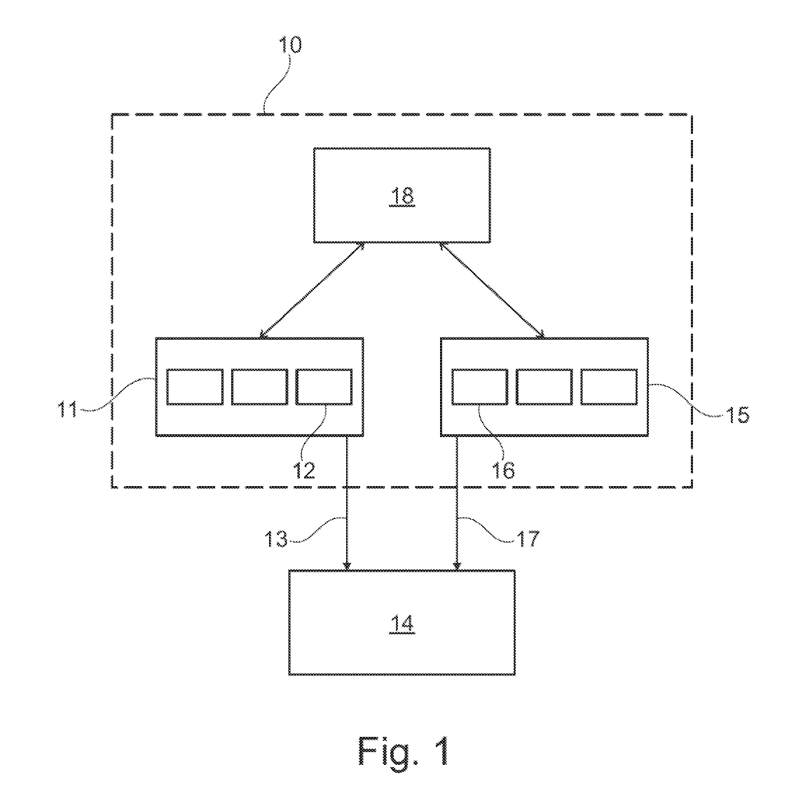

A schematic illustration of an example of an apparatus for testing the directional hearing of a test person according to a first embodiment of the invention is shown in FIG. 1. The apparatus 10 comprises an audio system 11 that comprises a plurality of audio output devices 12, in particular loudspeakers, for the output of test noises, tone signals, or other audio signals. Each audio signal 13 is output toward a position of a test person 14.

The apparatus 10 further comprises a light output system 15 with a plurality of light output devices 16 (i.e., lighting means for the output of light), whereby each light signal 17 is visibly output at least at the position of the test person 14. In the functional block diagram shown, the light output system 15 is illustrated next to the audio system 11. In practice, the light output devices 16 of the light output system 15 are in most cases arranged so that each light output device 16 is assigned, including spatially, to at least each audio output device 12 (or virtual location for producing a perceived output device location), in such a way that the test person 14 can check the direction of the audio signal 13 on the basis of the direction of the light signal 17.

The audio system 11 and the light output system 15 are connected to a control device 18, which is, for example, a programmable device with a processor and memory in which code portions of a suitable control software are stored, which are loaded by the processor in order to realize the functionality of the control device. The control device 18 arranges for the audio system 11 to output a plurality of audible audio signals one after another to the position of the test person 14 in such a way that, for the test person 14, an audio signal source of the respective audio signal is respectively located at selected ones of a plurality of different directions. On the other hand, the control device 18 arranges for the light output system 15 to assign at least one light output device 16 to each of the different directions in which the audio signal sources can be located for the test person 14, and to arrange for these, depending on the audio signal 13 being output at the particular time, to output an assigned light signal 17 at the position of the test person 14.

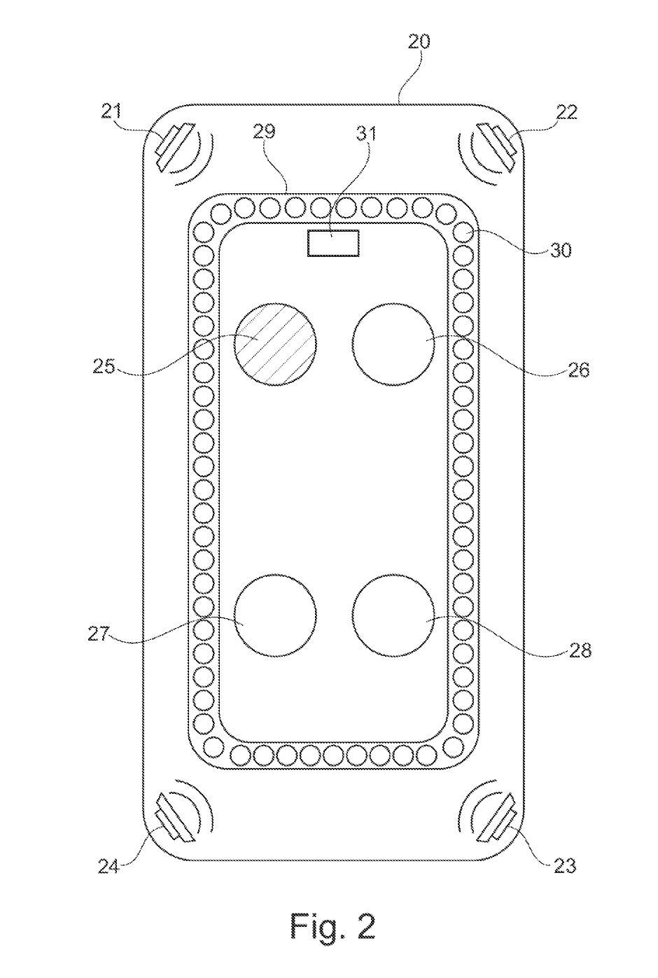

A schematic illustration of an example of a vehicle with an apparatus for testing the directional hearing of a test person according to a second embodiment of the invention is shown in FIG. 2. The interior cabin of the vehicle 20 has an audio system with, for example, four loudspeakers 21, 22, 23, 24 as audio output devices, and is designed as a directional audio system with which a virtual audio signal source is determined, for example based on wave-field synthesis, panning control, and/or other methods, and can be generated through appropriate drive of the loudspeakers 21, 22, 23, 24 in each direction with respect to the test person, in this case a person in the driver's seat 25 of the vehicle 20. In the example illustrated, the other seats in the interior of the vehicle 20 are unoccupied seats 26, 27, 28. As a light output system, the vehicle 20 has a ring of lights 29 with a plurality of, for example, light emitting diodes as lighting means or light output devices 30 in the interior, attached, for example around the roof, so that a light output device located at least approximately in the same direction can be assigned to the position of each virtual audio source. The control of the audio signal output and of the subsequent, assigned light signal output is achieved with a control device 31 connected to the audio system and the ring of lights 29.

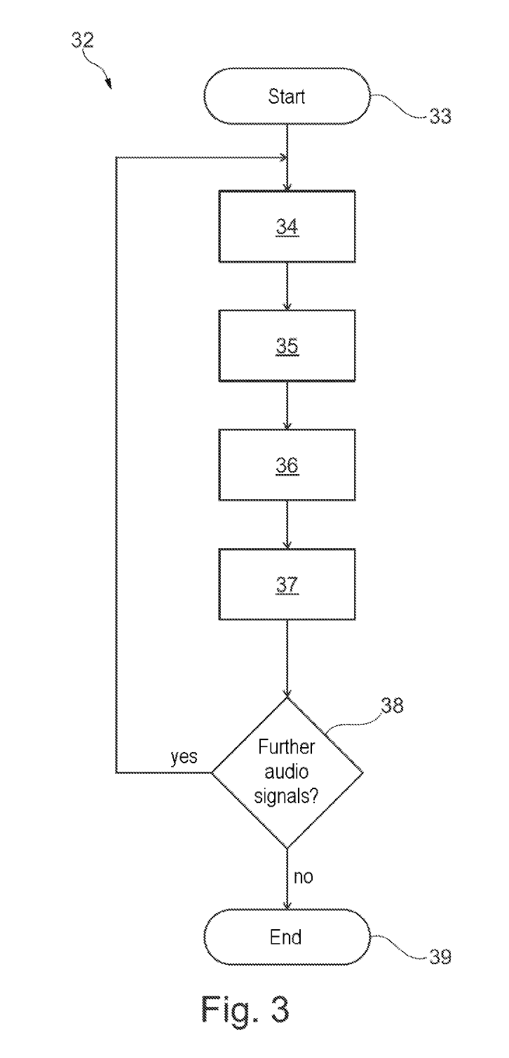

A schematic illustration of an example of a method for testing the directional hearing of a test person according to a second embodiment of the invention, and thereby a suitable operating method for the devices illustrated in FIG. 1 and FIG. 2, is explained in FIG. 3. The method 32 begins in a “Start” state 33, in which the device is placed into a state in which it can execute the method and, in particular, the control device is also appropriately initialized.

A selection 34 of a direction unknown for a test person relative to their position then takes place. The selection of the direction is predetermined by the properties of the audio system in use. All positions, or the angles relative to the alignment of the test person at his position, at which a (virtual or real) audio signal can be arranged by the audio system may be considered as possible directions.

In a next step, an output 35 of an audible audio signal that comes from a direction unknown, i.e. unexpected, for the test person is made with the audio system. The task of the test person here is to attempt to recognize the direction from which the audio signal comes.

In a further step, an output 36 of light, or of a light signal, at least also at the position of the test person, is made with a light output device of a light output system. The light output device is assigned here to the direction of the audio signal, so that, within the scope of the possibilities predetermined by the properties of the light output system and the audio system, the direction of the light signal output matches that of the audio signal. The test person can thereby check whether the direction of the source of the audio signal he has conjectured is correct. The output 36 of light here preferably takes place with a delay with respect to the output of the audio signal, so that the estimation of the direction from which the audio signal comes is not influenced by the light signal.

In the embodiment illustrated, a renewed output 37 of the audio signal and the simultaneous renewed output of the assigned light signal are furthermore subsequently provided, in order to reinforce the learning effect.

In the embodiment illustrated, a query 38 in particular then takes place as to whether further audio signals should be output, i.e., whether the test or the training should be continued. This can, for example, be controlled through an input facility by the test person or, for example, have been specified when the start state is initialized. It can, for example, be specified that a test sequence should comprise 2, 3 or 10 or another number of audio signals and associated light signals. If the query 38 reveals that a further audio signal should be output, the steps of the selection 34 of a direction again unknown for a test person relative to their position, the output 35 of an audio signal, the output 36 of light or of a light signal and, in the embodiment shown in FIG. 3, also the renewed output 37 of the audio signal and the simultaneous renewed output of the associated light signal, are repeated. If the query 38 reveals that no further audio signal should be output, the method ends at 39.

In another embodiment, it can be provided, in particular when a number of audio signals to be output is already specified, to execute the step of the selection 34 of an unknown direction only once, and to specify an entire test sequence even before the repetition of the other steps. In any case, a random generator can be used in the selection 34.

In a further embodiment it can be provided that in a simplified operating mode the audio and light signals are always output simultaneously.

In yet a further embodiment it can additionally be provided that, for example with the aid of video analysis, a gesture recognition of directions in which the test person points is carried out and, for example, that the query 38 only leads to a continuation of the test with an audio signal from another direction if the gesture recognition has shown that the test person has pointed in the correct direction from which the audio signal that has just been heard did come. It can in addition be provided here that a visual confirmation is displayed to the test person through the light output system if a direction is correctly recognized, e.g., flashing or changing the color to, for example, green, or otherwise indicating the error visually, for example through a color change to, for example, red.

Although the invention has been more closely illustrated and described in more detail through the preferred exemplary embodiments, the invention is not restricted by the disclosed examples, and other variations can be derived from this by the expert without going beyond the scope of protection of the invention.

It will be obvious that although method steps are described in accordance with a certain ordered sequence, they could in part be carried out in a sequence other than that described here. It will also be obvious that certain steps could be carried out simultaneously, that other steps could be added, or that certain steps described here could be omitted. The present descriptions are in other words provided for the purpose of clarifying particular forms of embodiments, and should not be taken as a restriction of the disclosed object.

Other embodiments can be used and structural or logical changes can be made without deviating from the scope of protection of the present invention. It will be obvious that the features of the different exemplary embodiments described above and below can be combined with one another except where specifically otherwise stated. The description is therefore not to be understood in a restrictive sense, and the scope of protection of the present invention is defined by the appended claims.

What is claimed is:

1. An apparatus for testing the directional hearing of a test person, comprising:
an audio system comprising a plurality of audio output devices for output of audible audio signals to a position of the test person;
a light output system comprising a plurality of light output devices for the output of light to the position of the test person; and
a control device connected to the audio system and to the light output system configured to:
activate the audio system to output a plurality of audible audio signals one after another to the position of the test person in such a way that, for the test person, each respective audio signal arrives from a respective one of a plurality of different directions; and
activate the light output system to output light indicative of a corresponding one of the plurality of different directions to at least the position of the test person, wherein the light output system is arranged to assign at least one of the light output devices to each of the plurality of different directions in which the audio signal source can be located for the test person, and to arrange for each respective activation of a light output device to be output at a particular time in relation to the respective audio signal.

2. The apparatus of claim 1, wherein the audio system is a directional audio system comprising means for generating the audio signal source as a virtual audio signal source through simultaneously driving the plurality of audio output devices to provide the plurality of different directions.

3. The apparatus of claim 2, wherein the control device is designed to generate the virtual audio signal source as a moving virtual audio signal source.

4. The apparatus of claim 1, wherein the audio system comprises means for selecting one of the plurality of audio output devices at a time as the audio signal source.

5. The apparatus of claim 1, wherein the plurality of audio outputs devices are loudspeakers of an audio entertainment system for the interior of a vehicle.

6. The apparatus of claim 5, wherein the plurality of light output devices comprises a row of lights in the interior of the vehicle.

7. The apparatus of claim 1, wherein the control device is designed to arrange for the light output system to output the light with a delay with respect to the audio signal being output at a respective time.

8. The apparatus of claim 1, wherein the control device is designed to specify the different directions for the audio signals to be output by the audio system one after another randomly.

9. The apparatus of claim 1, further comprising an evaluation device with a camera sensor and a gesture recognition module for recognizing a direction in which the test person is pointing.

10. A vehicle comprising:
an interior cabin having a seating position for a test person;
an audio system comprising a plurality of audio output devices for output of audible audio signals to the position of the test person;
a light output system comprising a plurality of light output devices for the output of light to the position of the test person; and
a control device connected to the audio system and to the light output system configured to:
activate the audio system to output a plurality of audible audio signals one after another to the position of the test person in such a way that, for the test person, each respective audio signal arrives from a respective one of a plurality of different directions; and
activate the light output system to output light indicative of a corresponding one of the plurality of different directions to at least the position of the test person, wherein the light output system is arranged to assign at least one of the light output devices to each of the plurality of different directions in which the audio signal source can be located for the test person, and to arrange for each respective activation of a light output device to be output at a particular time in relation to the respective audio signal.

11. The vehicle as claimed in claim 10, wherein the control device is configured to capture a driving state of the vehicle and to only permit an output of the audio signals and light signals when the driving state is stationary or in a self-controlling mode.

12. A method for testing the directional hearing of a test person, the method comprising the steps of:
selecting a direction unknown for a test person relative to their position;
outputting an audible audio signal that comes from the selected direction to the test person;
outputting a light signal to the position of the test person with a light output device of a light output system, wherein the light output device is assigned to the direction of the audio signal.

13. The method of claim 12, wherein the selecting and outputting steps are repeated at least once.

14. The method of claim 12, wherein the output of the light is delayed with respect to the output of the audio signal.

15. The method claim 12, further comprising a confirmation step wherein a renewed output of the same audio signal and simultaneous renewed output of the same light output are activated.

16. The method of claim 12, wherein the step of selecting the direction for outputting the audio signal is performed randomly.
